(19) United States
(12) Patent Application Publication
(10) Pub. No.: US 2008/0280963 A1
(43) Pub. Date: Nov. 13, 2008

(54) New amino-alkyl-amide derivatives as CCR3 receptor ligands

(75) Inventors: Agnes PAPPNE BEHR, Budapest (HU); Zoltan KAPUI, Budapest (HU); Peter ARANYI, Budapest (HU); Sandor BATORI, Budapest (HU); Veronika BARTANE BODOR, Budapest (HU); Lajos T. NAGY, (US); Mihalyne Santa, Mezotur (HU); Marton VARGA, Dunakeszi (HU); Endre MIKUS, Budapest (HU); Katalin URBAN-SZABO, Budapest (HU); Judit VARGANE SZEREDI, Budapest (HU); Tibor SZABO, Budapest (HU); Edit SUSAN, Dunakeszi (HU); Marianna KOVACS, Pomaz (HU)

Correspondence Address: ANDREA Q. RYAN;SANOFI-AVENTIS U.S. LLC, 1041 ROUTE 202-206, MAIL CODE: D303A, BRIDGEWATER, NJ, 08807, US

(73) Assignee: SANOFI-AVENTIS, Paris (FR)

(21) Appl. No.: 12/050,965

(22) Filed: Mar. 19, 2008

Related U.S. Application Data
(63) Continuation of application No. PCT/HU2006/000080, filed on Sep. 21, 2006.

(30) Foreign Application Priority Data
Sep. 22, 2005 (HU) P0500886

(51) Int. Cl. A61K 31/423; C07C 211/63; C07D 263/54; A61P 31/18; C07D 235/04; A61K 31/14

(52) U.S. Cl. 514/375; 564/289; 514/643; 548/217; 548/309.7; 514/394

(57) ABSTRACT

The invention relates to a compound of the general formula (I),

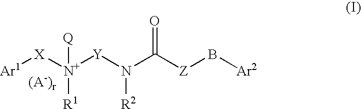

as defined herein which is useful for the treatment of a pathology in a patient wherein a CCR3 receptor plays a role in the development of the pathology, and pharmaceutical preparations containing such compound.

The invention is also directed to a process for preparing the compound of the general formula (I), and intermediate useful in the preparation.

### FIELD OF THE INVENTION

The present invention relates to the CCR3 receptor ligands of general formula (I), within them favourably antagonists and to the salts, solvates and isomers thereof, to the pharmaceutical compositions containing them, to the use of the compounds of the general formula (I) and their salts, solvates and isomers and to the preparation of the compounds of the general formula (I) and their salts, solvates and isomers.

### BACKGROUND OF THE INVENTION

Chemokines are small molecular weight (8-12 kDa) secreted polypeptides playing important regulatory role in the immune processes due to their leukocyte attracting (chemotactic) effect. They exert their effects through the chemokine receptors, which belong to the family of the G protein coupled receptors.

The CC chemokine receptors 3 (CCR3 receptors) are expressed by a number of inflammatory cells, like the basofils, mast cells, T lymphocytes, epithelial cells, dendritic cells, but in the greatest amount they can be found on the surface of the eosinofils.

The CCR3 receptor ligands belong to the family of the C—C chemokines. They have a number of selective and non-selective ligands. The selective ligands are the eotaxin, eotaxin-2 and the lately discovered eotaxin-3. The non-selective ligands are the RANTES, the monocyte chemotactic proteins (MCP-2, MCP-3, MCP-4) and the macrophage inhibitor protein (MIP-1). The best-characterized CCR3 ligand known from a long time is the eotaxin.

The eotaxin through the activation of the CCR3 receptors attracts selectively the eosinofils. Prior to an allergen provocation, the measured eotaxin level in the broncho-alveolar lavage fluidum of asthmatic patients was by 67 percent higher. On the effect of provocation a 2.4-fold increase of the epithelial and endothelial cells of the respiratory tract were found.

In the lung the eotaxin is produced in many cells. Following an allergen response, the most important eotaxin sources are the epithelial cells, but a great amount of eotaxin is produced by the fibroblasts of the lung, the smooth muscle cells and the endothelial cells of the respiratory tract, the alveolar macrophages and lymphocytes, and the eosinofils themselves.

Originally the data showed that the CCR3 receptors are to be found only in the eosinofil cells (Bertrand C P, Ponath P D., Expert Opin Investig Drugs. 2000 January; 9(1): 43-52), but on the basis of expression profiles it has been revealed that other inflammatory cells—although in smaller amount—also contain CCR3 receptors (Elsner J, Escher S E, Forssmann U., Allergy. 2004 December; 59(12): 1243-58). Thus, the CCR3 antagonists possess much wider effect, their activity is not limited to the eosinofils and consequently they can be considered much more valuable and effective targets in the treatment of asthmatic, allergic and inflammatory diseases.

Based on the above observations, CCR3 antagonists may possess important profilactic and therapeutic effects in the treatment of pathologies where in the development of the disease CCR3 receptors play a role. These diseases are characterized by the disorder of the leukocyte functions (activation, chemotaxis), there are numerous chronic inflammatory diseases among them, such as asthma, allergic rhinitis, atopic dermatitis, eczema, inflammatory bowel disease, ulcerative colitis, allergic conjunctivitis, arthritis, multiple sclerosis, Crohn's disease, HV-infection and diseases in conjunction with AIDS.

The CCR3 antagonists published to date in the literature are carbamide-, thiocarbamide derivatives (WO 01/09088, WO 02/059081) and/or compounds containing saturated cyclic amino group (WO 00/35451, U.S. Pat. No. 6,605,623, WO 01/98270, WO 03/004487, WO 03/018556, WO 2004/028530, WO 00/53600, WO 00/35876, WO 01/64216, WO 02/50064, WO 02/102775, GB 2373186, WO 03/082291, WO 2004/004731, WO 2004/058702, WO 2004/085423). The present invention relates to a new structural type of compounds, to the open-chain amino-alkyl-amide derivatives, representatives of these compounds are effective CCR3 receptor antagonists.

From the aspect of therapeutic use it is essential that the molecules do not bind, or bind only in case of very high concentration to other the CCR receptor subtypes.

Our aim was to prepare compounds of high antagonistic activity, and at the same time selective to the CCR3 receptor, i.e. which inhibit the CCR3 receptor in much smaller concentration as compared to other CCR receptors. Further aim was that the new compounds have stability, bioavailability, therapeutic index and toxicity values, which ensure its drugability. Additional aim was that the compounds, through their good enteric absorption could be applied orally.

### SUMMARY OF THE INVENTION

We have found that the compounds of general formula (I),

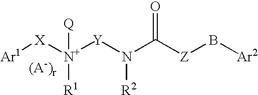

### (I)

where
Ar¹ stands for phenyl group, optionally substituted with one or more halogen atom;
X and Y independently mean straight or branched C₁₋₄ alkylene group, optionally substituted with one or more identical or non-identical straight or branched C₁₋₄ alkyl group;
Z means valence bond, or straight C₁₋₄ allylene group or straight C₂₋₄ alkenylene group, optionally substituted with one or more identical or non-identical straight or branched C₁₋₄ alkyl group;
B means valence bond, —S—, —SO—, SO₂—, or together with Z a straight C₂₋₄ alkylene group, optionally substituted with one or more identical or non-identical straight or branched C₁₋₄ alkyl group;
Q means straight or branched C₁₋₄ alkyl group, amino group or oxygen atom;
R¹ and R² independently mean hydrogen atom or straight or branched C₁₋₄ alkyl group;
Ar² stands for phenyl group, optionally substituted with halogen atom;
   5- or 6-membered heterocyclic ring containing one, two or three nitrogen atoms, or two nitrogen atoms and one oxygen atom, or one nitrogen atom and one oxygen atom, or one nitrogen atom and one sulphur atom, optionally substituted with one or more identical or non-identical substituents selected from the group consisting of straight or branched C₁₋₄ alkyl group, halogen atom, phenyl group—optionally substituted with one or more straight or branched C₁₋₄ alkyl group, halogen atom or benzyloxy group-, and oxo group;
   benzologue of these 5- or 6-membered heterocyclic ring group wherein the benzene ring may optionally be further substituted with one or more identical or non-identical substituents selected from the group consisting of halogen atom, straight or branched C₁₋₄ allyl group, straight or branched C₁₋₄ alkoxy group, hydroxyl group, trifluoromethyl group, nitro group, C₁₋₂ allylenedioxy group, amino group, amino group—substituted with one or two identical or non-identical straight or branched C₁₋₄ alkyl or benzyl group-, and sulfonyl group; or
   5-membered heterocyclic ring group containing one, two or three nitrogen atoms, or one nitrogen atom and one oxygen atom, or one nitrogen atom and one sulphur atom, condensed with a 6-membered heteroaromatic ring group containing one or two nitrogen atoms, optionally substituted with one or more identical or non-identical substituents selected from the group consisting of straight or branched C₁₋₄ alkyl group, straight or branched C₁₋₄ alkoxy group, hydroxyl group, amino group, and amino group—substituted with one or two identical or non-identical straight or branched C₁₋₄ alkyl group or benzyl group-;
A⁻ represents an anion;
r means 0 or 1;
   and their salts, solvates and isomers and the salts and solvates thereof, fulfill the above criteria.

### DETAILED DESCRIPTION OF THE INVENTION

The detailed meanings of the above substituents are as follows:

By a C₁₋₄ alkyl group we mean a saturated straight- or branched-chain aliphatic group of 1-4 carbon atom, such as methyl-, ethyl-, propyl-, isopropyl-, butyl-, isobutyl-, secondary butyl-, tertiary butyl group.

By a C₁₋₄ alkylene group we mean a —(CH₂)ₙ— group where the value of n is 1, 2, 3 or 4, such as a methylene-, ethylene-, propylene-, butylene group.

By a C₂₋₄ alkenylene group we mean an alkenylene group containing 1 double bound, e.g. a —CH═CH— or —CH₂—CH═CH-group.

By a C₁₋₄ alkoxy group we mean an —O-alkyl group—where the meaning of alkyl is as defined above-, such as methoxy-, ethoxy-, propoxy-, isopropoxy-, butoxy-, isobutoxy-, secondary butoxy-, tertiary butoxy group.

By a C₁₋₂ allylenedioxy group we mean an —O-alkylene-O— group—where the meaning of alkylene is as defined above-, such methylenedioxy-, ethylenedioxy group.

By halogen atom we mean chloro, fluoro, iodo or bromo atom.

By a 5- or 6-membered heterocyclic ring containing one, two or three nitrogen atoms we mean an unsaturated, saturated or partially saturated heterocyclic ring, for example pyrrole, imidazole, pyrazole, 1,2,3-triazole, 1,2,4-triazole, pyridine, pyrimidine, pyridazine, pyrazine 1,2,4-triazine, 1,3,5-triazine, 1,2,3-triazine, pyrrolidine, imidazolidine, [1,2,4]triazolidine, piperidine, piperazine, 2-imidazoline ring.

By a 5- or 6-membered heterocyclic ring containing one nitrogen atom and one oxygen or sulphur atom we mean an unsaturated, saturated or partially saturated heterocyclic ring, for example oxazole, isoxazole, thiazole, isothiazole, 1,2-oxazine, 1,3-oxazine, 1,4-oxazine, 1,2-thiazine, 1,3-thiazine, 1,4-thiazine, oxazolidine, thiazolidine, morpholine, thiomorpholine, 2-thiazoline, 2-oxazoline ring.

The heterocyclic ring containing two nitrogen atoms and one oxygen atom may be for example an oxadiazole ring.

By benzologue we mean derivatives condensed with benzene ring, for example indole, benzoxazole, benzthiazole, benzimidazole, quinoline, quinazoline, quinoxaline.

A derivative of a 5-membered heterocyclic ring—containing one, two or three nitrogen atoms, or one nitrogen atom and one oxygen atom, or one nitrogen atom and one sulphur atom—condensed with 6-membered heterocyclic rings—containing one or two nitrogen atom, may for example be a thiazolopyridine, triazolopyridine, thiazolopyrimidine, oxazolopyridine, 9H-purine, 3H-imidazopyridine.

By anion we mean pharmacologically acceptable anions, e.g. halogenide, tosylate, sulphate, phosphate anion.

By salts of the compounds of general formula (I) we mean salts given with inorganic and organic acids and bases. Preferable are the salts formed with pharmaceutically acceptable acids e.g. hydrochloric acid, sulphuric acid, ethanesulfonic acid, tartaric acid, fumaric acid, citric acid, and bases e.g. sodium hydroxide, potassium hydroxide, ethanolamine. The salts formed during the purification and isolation process, favourably with tetrafluoroboric acid and perchloric acid, are also subjects of the invention.

By solvates we mean solvates formed with various solvents, e.g. with water or ethanol.

By isomers we mean structural and optical isomers. Structural isomers may be tautomeric forms in equilibrium or isolated desmotrops, which are also subjects of the invention. The compounds of general formula (I) may contain one or more asymmetric carbon atom, thus they may be optical isomers, enantiomers or diastereoisomers. These enantiomers and diastereoisomers and the mixtures thereof, including the racemates are also subjects of the invention.

A favourable group of the compounds of general formula (I) is formed by the compounds, where
Ar¹ represents phenyl group, optionally substituted with one or more halogen atom;
X and Y independently mean straight C₁₋₄ alkylene group, optionally substituted with one or more identical or non-identical straight or branched C₁₋₄ alkyl group;
Z means valence bond, or a straight C₁₋₄ alkylene group—optionally substituted with one or more identical or non-identical straight or branched C₁₋₄ alkyl group-;
B means valence bond, —S—, —SO—, or —SO₂—, or together with Z a straight C₂₋₄ alkylene group-optionally substituted with one or more identical or non-identical straight or branched C₁₋₄ alkyl group-; Q means straight or branched C₁₋₄ allyl group, amino group or oxygen atom;
R¹ and R² independently mean hydrogen atom or straight or branched C₁₋₄ alkyl group;
A² stands for phenyl group;
   5- or 6-membered heterocyclic ring containing one, two, or three nitrogen atoms, or one nitrogen atom and one oxygen atom, or one nitrogen atom and one sulphur atom, optionally substituted with one or more straight or branched C₁₋₄ alkyl group;
   benzologue of these 5- or 6-membered heterocyclic ring group where the benzene ring may optionally be further substituted with one or more halogen atom, straight or branched C₁₋₄ allyl group; or
   5-membered heterocyclic ring containing two or three nitrogen atoms, or one nitrogen atom and one oxygen atom, or one nitrogen atom and one sulphur atom, condensed with a 6-membered heteroaromatic ring group containing one or two nitrogen atoms, optionally substituted with one or more amino group, and amino group—substituted with one or two identical or non-identical straight or branched C₁₋₄ alkyl group-;
A⁻ represents an anion; and
r means 0 or 1;
   and their salts, solvates and isomers and the salts and solvates thereof.

Especially favourable are the following compounds:
N-{3-[(3,4-Dichlorobenzyl)(methyl)nitroryl]propyl}-2-(6-methylbenzoxazol-2-ylsulfanyl)acetamide,
1-(3,4-Dichlorobenzyl)-1-methyl-1-[3-{[(6-methylbenzoxazol-2-ylsulfanyl)acetyl]-amino}propyl]diazanium tosylate,
   N-(3,4-Dichlorobenzyl)-N,N-dimethyl-3[(phenylacetyl)amino]propanaminium iodide, and their salts, solvates and isomers and the salts and solvates thereof.

The present invention relates furthermore to the pharmaceutical preparations containing the compounds of the general formula (I) or its isomers, salts or solvates, which are preferably oral preparations, but inhalable, parenteral and transdermal preparation also form a subject of the present invention. The above pharmaceutical preparations may be solid or liquid formulations, for example tablets, pellets, capsules, patches, solutions, suspensions or emulsions. The solid formulations, first of all the tablets and capsules are preferred.

The above pharmaceutical preparations are prepared by applying the usual excipients and technological operations.

The compounds of the general formula (I) according to the invention can be used for the treatment of pathologies where CCR3 receptors play a role in the development of the disease.

The compounds according to the present invention can favourably used in the treatment of diseases selected from asthma, allergic rhinitis, atopic dermatitis, eczema, inflammatory bowel disease, ulcerative colitis, allergic conjunctivitis, multiple sclerosis, Crohn's disease, HIV-infection and diseases in conjunction with AIDS.

A further subject of the invention is the use of the compounds of the general formula (I) for the treatment of the above pathologies. The suggested daily dose is 1-100 mg of the active component, depending on the nature and severity of the disease and the sex and weight of the patient.

A further subject of the invention is the preparation of the compounds of general formula (I) where in the formula Ar¹, X, Y, Z, B, Q, R¹, R², Ar², A⁻ and r have the meanings as defined above, and their salts, solvates and isomers.

Scheme 1. demonstrates the process of preparation of the compounds of general formula (I).

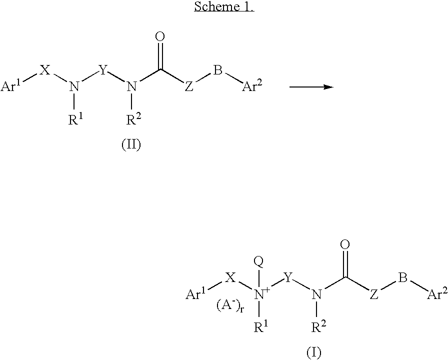

In the process according to the invention
a.) for the preparation of compounds of general formula (I) where Q represents a straight or branched C₁₋₄ alkyl group and the meanings of Ar¹, X, Y, Z, B, R¹, R², Ar², and A⁻ are as defined above and the value of r is 1,
   a compound of general formula (II),

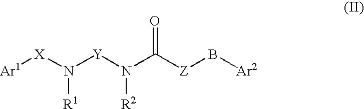

where the meanings of Ar¹, X, Y, Z, B, R¹, R², Ar² are as defined above is reacted with an alkylating agent suitable to introduce the group Q, or
b.) for the preparation of compounds of general formula (I) where Q represents amino group and the meanings of Ar¹, X, Y, Z, B, R¹, R², Ar², and A are as defined above and the value of r is 1, a compound of general formula (II), where the meanings of Ar¹, X, Y, Z, B, R¹, R², Ar² are as defined above, is reacted with O-tosylhydroxylamine, or
c.) for the preparation of where Q represents oxygen and the meanings of Ar¹, X, Y, Z, B, R¹, R², Ar² are as defined above and the value of r is 0, a compound of general formula (II), where the meanings of Ar¹, X, Y, Z, B, R¹, R² and Ar² are as defined above, is oxidized and
   if desired the substituents and the anion of the compound of general formula (I) thus obtained are transformed into each other by using known methods and/or the resulting compound of general formula (I) is transformed into its salt or solvate, or liberated from its salt or solvate and/or resolved into its optically active isomers, or the optically active isomer is transformed into the racemic compound and if desired the structural isomers are separated from each other.

In a preferred embodiment of process a.) according to the invention, alkylation is carryied out preferably with alkyl sulphates, allyl phosphates or alkyl halogenides, most preferably with alkyl iodides, in inert solvents. As inert solvent a halogenated hydrocarbon, such as dichloromethane, chloroform, tetrahydrofuran, acetonitrile, preferably acetonitrile is used. The reaction is performed at 0° C.-50° C.

In process version b.) the reaction is preferably carried out in inert solvent at a temperature between 0° C.-50° C. As inert solvent a halogenated hydrocarbon, such as dichloromethane, chloroform, tetrahydrofuran, acetonitrile, preferably acetonitrile is used.

In process version c.) known oxidants, such as hydrogen peroxide, potassium permanganate, preferably meta-chloroperbenzoic acid are used as oxidizing agent The reaction is preferably carried out at a temperature between 0° C.-30° C.

The new starting materials of general formula (II) and the processes for their preparation are described in Hungarian patent applications PO500877, PO500878 and PO500879 and in their corresponding PCT applications PCT/HU2006/00077; PCT/HU2006/00078; PCT/HU2006/00079.

Scheme 2. demonstrates one possible way of preparing compounds of the general formula (II)

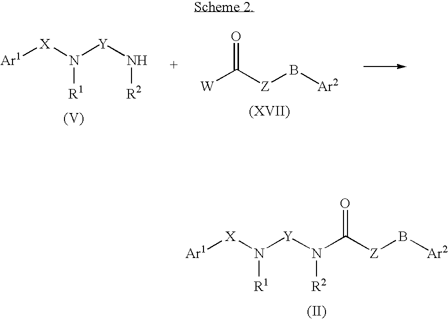

In the process shown in Scheme 2. a diamine of the general formula (V),

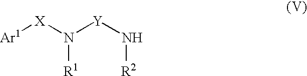

where the meanings of Ar¹, X Y, R¹ and R² are as defined above, is reacted with a carboxylic acid derivative of general formula (XVII),

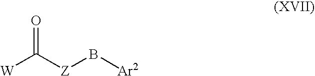

where the meanings of Ar², Z and B are as defined above and W stands for halogen atom, hydroxyl group, —OR¹¹-group, wherein R¹¹ means C₁₋₄-allyl group or —O—CO-Z-B—Ar²-group, wherein the meaning of Z, B and Ar² are as defined above, and if desired, the substituents of the compound of general formula (II) thus obtained are transformed into each other by using known methods and/or the resulting compound of general formula (II) is transformed into its salt or solvate, or liberated from its salt or solvate and/or resolved into its optically active isomers, or the optically active isomer is transformed into the racemic compound and if desired the structural isomers are separated from each other.

In a preferred embodiment of the process the acid of general formula (XVII)—where W stands for hydroxyl group—is transformed into an acid chloride, by using acid chloride-forming reagents, favourably thionyl chloride, and the resulting acid chloride is reacted in an inert solvent, like dichloromethane, chloroform, or ethyl acetate, with the amine of general formula (V), in the presence of a base, like triethylamine, or in pyridine, or in aqueous alkali solution, at room temperature or under reflux conditions.

In another preferred method the acid of general formula (XVII)—where W stands for hydroxyl group—is reacted with the amine of general formula (V), in the presence of an activating agent. Activation of the carboxylic acid may tale place via mixed anhydride intermediates, by using e.g pivalyl chloride (M. T. Leplawy: Tetrahedron 1960, 11, 39), ethyl chloroformate (T. Wieland: J. Liebigs Ann. Chem. 1951, 572, 190), isobutyl chloroformate (J. R. Vaughan: JACS. 1951, 73, 3547) or dicyclohexylcarbodiimide (DCC) (R. Arshady: J. Chem. Soc. Perlcin Trans. 1, 1981, 529 or D. Hudson: J. Org. Chem. 1988, 53, 617) in an inert solvent, e.g. in dichloromethane, chloroform, tetrahydrofuran, acetonitrile, in the presence of an acid binding tertiary amine, e.g. triethylamine, N-methylmorpholine, at a temperature of −10° C. to 25° C.

The activation can furthermore be accomplished by use of carbonyldiimidazole (H. A. Staab: Lieb. Ann. Chem.: 1957, 609, 75), in an inert solvent, preferably in dichloromethane, chloroform, tetrahydrofuran, acetonitrile or in the mixture thereof or with benzotriazol-1-yl-oxy-tripyrrolidinophosphonium hexafluorophosphate (PyBOP), in an inert solvent (J. Corte: Tetrahedron Lett. 31, 1990, 205).

If the compound of the general formula (XVII) is a carboxylic acid ester, where in the formula W means an OR¹¹-group, the reaction can be carried out by one of the methods known in the literature, preferably at 100° C.-150° C., without solvent, in melt.

If the compound of the general formula (II) is a racemic compound, the separation of the enantiomers can be accomplished by chiral preparative column chromatography or by another known method suitable for the resolution of compounds of basic character.

The diamines of general formula (V) can be prepared by different methods depending on the nature of the substituents R¹, R², X and Y.

Scheme 3. presents the preparation of those compounds belonging to general formula (V) where in the formula R² stands for hydrogen atom, Y stands for 1,3-propylene, 1-methyl-1,3-propylene, 2-methyl-1,3-propylene or 1,4-butylene (R⁶ and R⁷ independently from each other represents hydrogen atom or methyl group, p is 0 or 1), and the meanings of Ar¹ and X are as defined above.

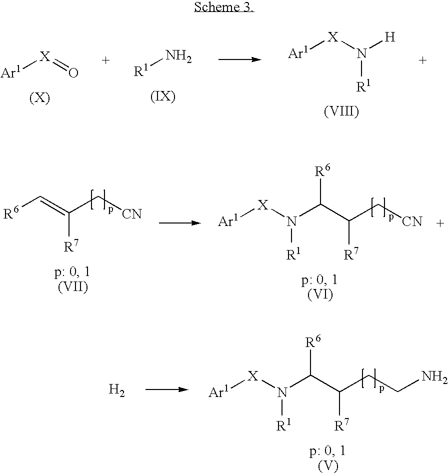

The compounds of the general formula (VIII) can be prepared by methods known in the literature starting from the oxo compounds (aldehydes or ketones) of the general formula (X) by reductive amination with the amines of general formula (IX) in alcoholic medium, in the presence of sodium cyanoborohydride (Holzgrabe U.: Arch. Pharm. 1987, 320, 7, 647-654), or by catalytic hydrogenation (Elslager E. F.: J. Med. Chem. 1981, 24, 2, 140-145), or with sodium borohydride in aqueous alcohol medium (Simig Gy.: J. Chem. Soc Perkin Trans. 1. 1992, 13, 1613-16). The compounds of the general formula (IX) are commercially available. The aldehydes of general formula (X) are commercially available or can be prepared by methods known in the literature. The compounds of general formula (VI) can be prepared from the compounds of general formula (VIII) with the alkene-cyanides of the general formula (VII) by literature analogies (King M. et al: JACS. 1946, 68, 1468, or Surrey et al: JACS. 1956, 78, 2573). The cyanides of the general formula (VII) are commercially available. The diamines of the general formula (V) can be obtained by catalytic hydrogenation of the cyanides of general formula (VI) by literature analogies, in alcohol or hexane solution, in the presence of ammonia and Raney nickel or rhodium catalyst, in a given case under pressure (Shapiro et al: JACS. 1959, 81, 3083-84, and Roufos I.: J. Med. Chem. 1996, 39, 7, 1514).

The diamines of the general formula (V), where in the formula the meaning of Y is ethylene group, R² stands for hydrogen atom and the meanings of Ar¹ and X are as defined above, can be prepared as shown in Scheme 4.,

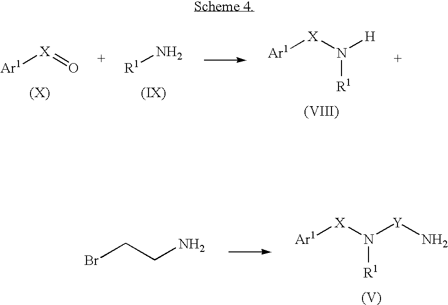

from the amines of the general formula (VIII) with 2-bromoethylamine, by literature analogy, in hot aqueous solution (Arz. Forsch. 1975, 25, 1853-58).

The diamines of the general formula (V), where R² stands for hydrogen atom, Y for 3-methylpropylene group and the meanings of Ar¹ and X are as defined above, can be prepared as shown in Scheme 5.

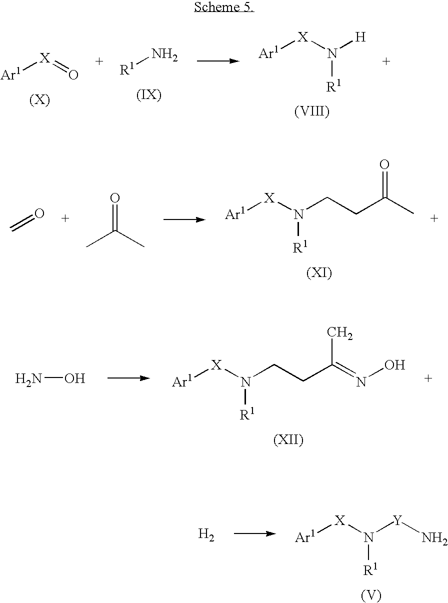

The compounds of general formula (XI) are obtained by Mannich condensation from the amines of general formula (VIII) with paraformaldehyde and acetone. By literature analogy, the reaction can be performed in i-propanol under reflux conditions (JACS. 1959, 81, 2214-18). The oximes of general formula (XII) are prepared from the compounds of general formula (XI) with hydroxylamine, by literature analogies, in aqueous i-propanol solution (JACS. 1959, 81, 2214-18). The amine of general formula (V) is prepared by literature analogy from the oxime of general formula (XII) by catalytic hydrogenation in the presence of Raney-Nickel catalyst, in ethanolic ammonia solution.

Scheme 6. demonstrates the preparation of the compounds of general formula (V) where R¹ and R² represents methyl group and the meanings of Ar¹, X and Y are as defined above.

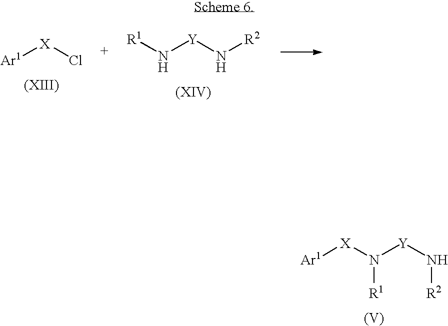

The compounds of the general formula (V) can be obtained by reacting the commercially available halogenides of the general formula (XI) with the N,N′-dimethylaminoalkyl compounds of general formula (XIV), in inert solvents, preferably in acetonitrile, in the presence of an acid binding organic amine.

The compounds of the general formula (X), where X represents 1,3-propylene group and the meaning of Ar¹ is as defined above, can be obtained as presented in Scheme 7.

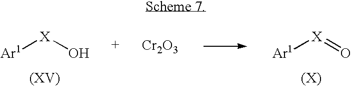

by analogies in the literature (J. Org. Chem. 2002, 67, 25, 8758-8763), from the appropriate alcohols of general formula (XV) by oxidation with pyridinium chlorochromate in inert solvent, preferably in dichloromethane.

The ketones of general formula (X), where X represents 3-methylpropylene group, can be prepared by the method shown in Scheme 8.,

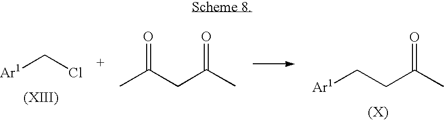

by analogies in the literature (Powel et al: JACS. 2004, 126, 25, 7788-89), by heating the commercially available benzylchlorides of general formula (XI) with pentane-2,4-dione in alcohol solution under reflux conditions, in the presence of potassium carbonate.

The compounds of the general formula (XVII) are commercially available or can be prepared by the methods known in the literature.

Further details of invention are demonstrated by the examples, without limiting the invention to the examples.

### EXAMPLE 1

### N-(3,4-Dichlorobenzyl)-N,N-dimethyl-3[(phenylacetyl)amino]propanaminium iodide

In the general formula (I) Ar¹ represents 3,4-dichlorophenyl group, X and Z stands for methylene group, Q for methyl group, R¹ for methyl group, Y for 1,3-propylene group, R² for hydrogen atom, B means a valence bond, Ar² means phenyl group, A represents iodide anion, r means 1.

### a.) N-(3,4-Dichlorobenzyl)-N-methyl-3[(phenylacetyl)amino]propane

To the solution of 0.24 g (1 mmol) N-(3,4-dichlorobenzyl)-N-(methyl)propane-1,3-diamine in 2 ml chloroform, 0.06 g (1.5 mmol) sodium hydroxide in 1 ml water is added, then 0.15 g (1 mmol) phenylacetyl chloride in 1 ml chloroform, dropwise under cooling. The reaction mixture is stirred at room temperature for 3 hours. The solvent is evaporated, the aqueous residue is extracted with 3×20 ml ethyl acetate, the organic phase is dried over sodium sulphate, filtered and evaporated to obtain 0.36 g title compound LC/MS[MH⁺]=365 (C₁₉H₂₂Cl₂N₂O 365.30)

### b.) N-(3,4-Dichlorobenzyl)-N,N-dimethyl-3-[(phenylacetyl)amino]propane aminium iodide

0.11 g (0.3 mmol) N-(3,4-dichlorobenzyl)-N-methyl-3 [(phenylacetyl)amino]propane is dissolved in 2 ml acetonitrile, to the solution 0.42 g (0.3 mmol) methyl iodide in 1 ml acetonitrile is added dropwise, at room temperature and the mixture is stirred for 14 hours. The solvent is evaporated, the residue is treated with ether, the solid material is filtered off and washed with ether to obtain 0.15 g title compound. Mp: 128-130° C.

### EXAMPLE 2

### 3-(Benzoylamino)-N-(3,4-dichlorobenzyl)-N,N-(dimethyl)propane-1-aminium iodide

In the general formula (I) Ar¹ represents 3,4-dichlorophenyl group, X stands for methylene group, R¹ for methyl group, Q for methyl group, Y for 1,3-propylene group, R² for hydrogen atom, Z and B represent a valence bond, Ar² means phenyl group, A⁻ represents iodide anion, r means 1.

Following the procedure as described in Example 1. b.) and starting from N-(3,4-dichlorobenzyl)-N-methyl-3-(benzoylamino)propane, 0.13 g title compound is obtained.

Mp: 59-62° C.

### EXAMPLE 3

### N-{3-[(3,4-Dichlorobenzyl)(methyl)nitroryl]propyl}-2-(6-methylbenzoxazol-2-ylsulfanyl)acetamide

In the general formula (I) Ar¹ represents 3,4-dichlorophenyl group, X and Z stand for methylene group, R¹ for methyl group, Q means O⁻, Y stands for 1,3-propylene group, R² for hydrogen atom, B represent a sulphur atom, Ar² means 6-methylbenzoxazol-2-yl group, r means 0.

To the solution of 0.27 g (0.6 mmol) N-{3-[(3,4-dichlorobenzyl)(methyl)amino]propyl}-2-(6-methylbenzoxazol-2-yl)sulfanyl]acetamide in 6 ml dichloromethane 0.11 g (0.66 mmol) meta-chloroperbenzoic acid is added under ice-water cooling and the mixture is stirred for 1 hour. The acid is neutralized with solid potassium carbonate, the precipitated salts are filtered off, the dichloromethane solution is evaporated. The residue is purified by column chromatography using chloroform-methanol 9:1 mixture as eluent. Thus, 100 mg title compound is obtained in the form of crystals. Mp: 89-90° C.

### EXAMPLE 4

### N-{3-[(3,4-Dichlorobenzyl)(methyl)nitroryl]propyl}-2-(1-methyl-1H-benzimidazol-2-ylsulfanyl)acetamide

In the general formula (I) Ar¹ represents 3,4-dichlorophenyl group, X and Z stand for methylene group, R¹ for methyl group, Q means O⁻, Y stands for 1,3-propylene group, R² for hydrogen atom, B for sulphur atom, Ar² means 1-methylbenzimidazol-2-yl group, r means 0.

### a.) N-{3-[(3,4-Dichlorobenzyl)(methyl)amino]propyl}-2-(1-methyl-1H-benzimidazol-2-ylsulfanyl)acetamide

### a/1.) Methyl-(1-methyl-1H-benzimidazol-2-ylsulfanyl)acetate

To the solution of 1.16 g (11 mmol) thioglycolic acid methyl ester in 14 ml chloroform, the solution of 1.2 g (12 mmol) triethylamine and 1.33 g (8 mmol) 2-chloro-1-methyl-1H-benzimidazol in 10 ml chloroform is added. The reaction mixture is heated at 60° C.—on for 20 hours. The chloroform solution is washed with water, with diluted potassium hydrogensulfate solution and with water, dried over sodium sulphate and evaporated. The residue is purified by column chromatography using hexane-ethyl acetate 2:1 mixture as eluent. The precipitated crystals are filtered off. Thus, 0.52 g title compound is obtained. LC/MS[MH⁺]=237 (C₁₁H₁₂SN₂O₂ 236.29)

### a/2.) N-{3-[(3,4-Dichlorobenzyl)(methyl)amino]propyl}-2-(1-methyl-1H-benzimidazol-2-ylsulfanyl)acetamide

The mixture of 0.52 g (2.2 mmol) methyl (1-methyl-1H-benzimidazol-2-ylsulfanyl)acetate and 0.61 g (2.5 mmol) N-(3,4-dichlorobenzyl)-N-(methyl)propane-1,3-diamine is heated and stirred at 100° C. for 1 hour. The melt is purified by column chromatography using chloroform as eluent. Thus, 350 mg title compound is obtained in the form of an oil. LC/MS[MH⁺]=451 (C₂₁H₂₄Cl₂N₄OS 451.42).

### b.) N-{3-[(3,4-Dichlorobenzyl)(methyl)nitroryl]propyl}-2-(1-methyl-1H-benzimidazol-2-ylsulfanyl)acetamide

To the solution of 0.68 g (0.15 mmol) N-{3-[(3,4-dichlorobenzyl)(methyl)amino]propyl}-2-(1-methyl-1H-benzimidazol-2-ylsulfanyl)acetamide in 1.5 ml dichloromethane 0.03 g (0.17 mmol) meta-chloroperbenzoic acid is added under ice-water cooling and the mixture is stirred for 1 hour. The acid is neutralized with solid potassium carbonate, the precipitated salts are filtered off, the dichloromethane solution is evaporated. The residue is purified by column chromatography using chloroform-methanol 4:1 mixture as eluent. Thus, 53 mg title compound is obtained in the form of crystals. Mp.: 106-110° C.

### EXAMPLE 5

### 1-(3,4-Dichlorobenzyl)-1-methyl-1-[3-{[(6-methylbenzoxazol-2-ylsulfanyl)acetyl amino}propyl]diazanium tosylate

In the general formula (I) Ar¹ represents 3,4-dichlorophenyl group, X and Y stand for methylene group, R¹ for methyl group, Q means amino group, Y stands for 1,3-propylene group, R² for hydrogen atom, B for sulphur atom, Ar² means 6-methylbenzoxazol-2-yl group, A⁻ represents tosylate anion, r means 1.

To the solution of 0.08 g (0.44 mmol) O-tosylhydroxylamine in 9 ml dichloromethane under ice-water cooling the solution of 0.18 g (0.4 mmol) N-{3-[(3,4-dichlorobenzyl)(methyl)amino]propyl}-2-(6-methylbenzoxazol-2-ylsulfanyl)acetamide in 5 ml dichloromethane is added dropwise. The reaction mixture is stirred for 30 minutes under cooling and for 2 hours at room temperature. The precipitate is filtered off, washed with dichloromethane. Thus, 0.15 g title compound is obtained. Mp: 112-114° C.

### EXAMPLE 6

In known methods the tablet of the following composition is prepared:

| | |
|---|---|
| Active component: | 40 mg |
| Lactose: | 35 mg |
| Avicel: | 21 mg |
| Crospovidone: | 3 mg |
| Magnesium stearate: | 1 mg |

### EXAMPLE 7

### A.) Human Recombinant CCR3 Receptor (hr-CCR3) Binding Assay

The CCR3 receptor antagonist effect of the compounds of general formula (I) was examined on eotaxin binding test on hCCR3 receptor expressing recombinant K562 and RBL2H3 cells. To the tests Eotaxin labelled with radioactive iodine ¹²⁵I-(2200 Ci/mmol) was used.

In the assay 200000 cells are incubated in the presence of 0.11 nM ¹²⁵I-Eotaxin, incubation: 60 minutes at 37° C. Composition of the assay buffer: RPMI-1640 medium, pH=7.6 (GIBCO), [containing 80 mg CHAPS, 500 BSA (protease free), 100 mg Gelatine, 3 ml 25 mM HEPES in 100 ml RPMI]. The test compounds are dissolved in DMSO, the stock solution is diluted with the assay buffer. The final DMSO concentration is not more than 1%. The assays are performed in deep-well plates. The cells are incubated with the test compounds for 15 minutes, then the labelled eotaxin is added. The non-specific binding is determined in the presence of 200 nM non-labelled eotaxin. After 1 hour of incubation, 500 μl ice-cold assay buffer containing 0.5 M NaCl solution is added. The reaction is terminated by centrifugation in plate centrifuge (JUAN) at 3600 g for 6 minutes. The supernatants are poured off by turning the plates in upside-down position. The remaining droplets were blotted with tissue paper. For solubilization 200 μl 0.5 M NaOH solution is added to the pellets. After 1 hour of solubilization at room temperature the radioactivity of 150 μl solubilized solution is counted in gamma counter (1470 Wizard, Wallac).

The radioactivity of the solution is in direct ratio with the number of the receptors of the cells, with the amount of the bound ¹²⁵I-Eotaxin and with the activity of the tested antagonist.

The specific binding is calculated as the difference between the total and the non-specific bindings. The activity of the compounds is calculated from the specific binding and from the binding measured in the presence of the antagonist molecule.

The activity of the compounds is characterized with the IC₅₀ value.

### B.) Investigation of Ca²⁺ Mobilization in hCCR3-RBL and hCCR3 K562 Cells

HCCR3-K562 and hCCE3-RBL2H3 cells in 40000 cells/well density (number of cells in one well of the microplate) are cultured for 24 hours. The cells are washed and loaded with calcium indicator dye (Calcium Plus assay Kit, Molecular Devices). The cells are incubated in the presence of the dye for 60 minutes while loading takes place. The dye is a fluorescent calcium indicator, which sensitively indicates the intracellular calcium concentration. The intracellular calcium concentration is in direct ratio with the fluorescent signal of the sample. The experiments are performed in a BMG NOVOSTAR apparatus, at exitation and emission wavelengths.

The selective agonists used in the experiments are:

Eotaxin

Eotaxin-2

Eotaxin-3

RANTES

Following the addition of the selective agonist, the intracellular calcium concentration in the cells significantly increases which can be monitored with the help of the fluorescent signal. In the experiments an agonist concentration is used which causes a 75% calcium signal compared to the maximum attainable signal.

Antagonists are added 15 minutes before the agonist treatment.

The change of the fluorescent signal is monitored for 30 seconds, during that period the process takes place.

The intensity of the maximum signal following the addition of the agonist is compared with the calcium signal obtained after the addition of the same agonist, but in the presence of the inhibitor.

The activity of the compounds is characterized with the IC₅₀ values.

On the basis of tests A and B the compounds of general formula (I) were found biologically active. The most potent compounds are the compounds of general formula (I) according to claim 2, which form a narrower group of the compounds of general formula (I) according to claim 1. Their IC₅₀ values are in the range of 0.5 nM to 500 nM. Of these compounds, the especially favoured molecules have IC₅₀ values between 0.5 nM and 15 nM.

1. A compound of the general formula (I), 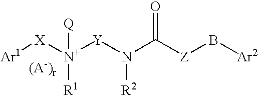
(I)
wherein
Ar¹ stands for phenyl group, optionally substituted with one or more halogen atom;
X and Y independently mean straight or branched C₁₋₄ alkylene group, optionally substituted with one or more identical or non-identical straight or branched C₁₋₄ alkyl group;
Z means valence bond, or straight C₁₋₄ alkylene group or straight C₂₋₄ alkenylene group, optionally substituted with one or more identical or non-identical straight or branched C₁₋₄ alkyl group;
B means valence bond, —S—, —SO—, or —SO₂—, or together with Z a straight C₂₋₄ alkylene group, optionally substituted with one or more identical or non-identical straight or branched C₁₋₄ alkyl group;
Q means straight or branched C₁₋₄ alkyl group, amino group or oxygen atom;
R¹ and R² independently mean hydrogen atom or straight or branched C₁₋₄ alkyl group;
Ar² stands for phenyl group, optionally substituted with halogen atom; 5- or 6-membered heterocyclic ring group containing one, two or three nitrogen atoms, or two nitrogen atoms and one oxygen atom, or one nitrogen atom and one oxygen atom, or one nitrogen atom and one sulphur atom, optionally substituted with one or more identical or non-identical substituents selected from the group consisting of straight or branched C₁₋₄ alkyl group, halogen atom, phenyl group-optionally substituted with one or more straight or branched C₁₋₄ alkyl group, halogen atom or benzyloxy group-, and oxo group;
benzologue of the 5- or 6-membered heterocyclic ring group wherein the benzene ring may optionally be further substituted with one or more identical or non-identical substituents selected from the group consisting of halogen atom, straight or branched C₁₋₄ alkyl group, straight or branched C₁₋₄ alkoxy group, hydroxyl group, trifluoromethyl group, nitro group, C₁₋₂ alkylenedioxy group, amino group, amino group—substituted with one or two identical or non-identical straight or branched C₁₋₄ alkyl or benzyl group-, and sulfonyl group; or
5-membered heterocyclic ring group containing one, two or three nitrogen atoms, or one nitrogen atom and one oxygen atom, or one nitrogen atom and one sulphur atom, condensed with a 6-membered heteroaromatic ring group containing one or two nitrogen atoms, optionally substituted with one or more identical or non-identical substituents selected from the group consisting of straight or branched C₁₋₄ alkyl group, straight or branched C₁₋₄ alkoxy group, hydroxyl group, amino group, and amino group—substituted with one or two identical or non-identical straight or branched C₁₋₄ allyl group or benzyl group-;
A⁻ represents an anion; and
r means 0 or 1; or
a salt, solvate or isomer thereof, salt of the solvate or isomer thereof, or solvate of the isomer thereof.

2. The compound of the general formula (I) according to claim 1, wherein
Z means valence bond, or a straight C₁₋₄ alkylene group optionally substituted with one or more identical or non-identical straight or branched C₁₋₄ alkyl group;
Ar² stands for phenyl group;
5- or 6-membered heterocyclic ring group containing one, two, or three nitrogen atoms, or one nitrogen atom and one oxygen atom, or one nitrogen atom and one sulphur atom, optionally substituted with one or more straight or branched C₁₋₄ alkyl group;
benzologue of the 5- or 6-membered heterocyclic ring group wherein the benzene ring may optionally be further substituted with one or more halogen atom, straight or branched C₁₋₄ alkyl group; or
5-membered heterocyclic ring containing two or three nitrogen atoms, or one nitrogen atom and one oxygen atom, or one nitrogen atom and one sulphur atom, condensed with a 6-membered heteroaromatic ring group containing one or two nitrogen atoms, optionally substituted with one or more amino group, or amino group—substituted with one or two identical or non-identical straight or branched C₁₋₄ alkyl group-; and
r means 0 or 1; or
a salt, solvate or isomer thereof, salt of the solvate or isomer thereof, or solvate of the isomer thereof

3. The following compound according to claim 1 selected from the group consisting of:
N-{3-[(3,4-Dichlorobenzyl)(methyl)nitroryl]propyl}-2-(6-methylbenzoxazol-2-ylsulfanyl)acetamide,
1-(3,4-Dichlorobenzyl)-1-methyl-1-[3-{[(6-methylbenzoxazol-2-ylsulfanyl)acetyl]-amino}propyl]diazanium tosylate, and
N-(3,4-Dichlorobenzyl)-N,N-dimethyl-3[(phenylacetyl)amino]propanaminium iodide, or a salt, solvate or isomer thereof, salt of the solvate or isomer thereof, or solvate of the isomer thereof

4. A process for the preparation of the compound of the general formula (I) according to claim 1 or a salt, solvate or isomer thereof comprising,
a) for preparing the compound of the general formula (I) where Q represents straight or branched C₁₋₄ alkyl group, and r is 1, alkylating a compound of the general formula (II), 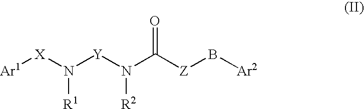
wherein Ar¹, X, Y, Z, B, R¹, R² and Ar² are as defined in claim 1, with an alkylating agent, suitable to introduce group Q, or
b) for preparing the compound of the general formula (I) wherein Q represents amino group, and r is 1, reacting the compound of the general formula (II), with O-tosylhydroxylamine, or
c) for preparing the compound of the general formula (I) wherein Q represents oxygen atom, and r is zero, oxidizing the compound of the general formula (II) with an oxidizing agent, and
optionally transforming a substituent of the compound of the general formula (I) thus obtained according to step a), b) or c) into another by using a known method and/or the resultant compound of the general formula (I) obtained according to step a, b or c is transformed into a salt or solvate thereof, or liberated from a salt or solvate thereof and/or resolved into an optically active isomer, or the optically active isomer is transformed into the racemic compound and if desired separating structural isomers from each other.

5. The process according to claim 4, pathway a), wherein the alkylating agent is an alkyl sulphate, alkyl phosphate, allyl halogenide.

6. The process according to claim 5, wherein the alkyl halogenide is alkyl iodide.

7. The process according to claim 4, pathway b), wherein the oxidizing agent is hydrogen peroxide, potassium permanganate, or meta-chloroperbenzoic acid.

8. The process according to claim 4, pathways a), b) or c), wherein the reaction is carried out in an inert solvent.

9. A pharmaceutical preparation wherein it contains one or more of the compounds of the general formula (I) according to claim 1, or a salt, solvate or isomer thereof, salt of the solvate or isomer thereof, or solvate of the isomer thereof and one or more excipients used in the pharmaceutical industry.

10. The pharmaceutical preparation according to claim 9, wherein the one or more compounds of the general formula (I) is/are selected from N-{3-[(3,4-Dichlorobenzyl)(methyl)nitroryl]propyl}-2-(6-methylbenzoxazol-2-ylsulfanyl)acetamide, 1-(3,4-Dichlorobenzyl)-1-methyl-1-[3-{[(6-methylbenzoxazol-2-ylsulfanyl)acetyl]-amino}propyl]diazanium tosylate, and N-(3,4-Dichlorobenzyl)-N,N-dimethyl-3[(phenylacetyl)amino]propanaminium iodide.

11. A method of treatment of a pathology in a patient wherein a CCR3 receptor plays a role in the development of the pathology comprising administering to the patient a pharmaceutically effective amount compound of the general formula (I) according to claim 1.

12. The method according to claim 11 wherein the pathology is selected from asthma, allergic rhinitis, atopic dermatitis, eczema, inflammatory bowel disease, ulcerative colitis, allergic conjunctivitis, multiple sclerosis, Crohn's disease, HIV-infection and diseases in conjunction with AIDS.
